(19) United States
(12) Patent Application Publication
(10) Pub. No.: US 2003/0199449 A1
(43) Pub. Date: Oct. 23, 2003

(54) Combination of ablation and controlled drug delivery for the treatment of cancer

(76) Inventors: Peter J. Tarcha, Lake Villa, IL (US); John R. Haaga, Chagrin Falls, OH (US); Nick T. Stowe, Lakewood, OH (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/390,971

(22) Filed: Mar. 18, 2003

Related U.S. Application Data
(60) Provisional application No. 60/374,036, filed on Apr. 19, 2002.

(51) Int. Cl. A61K 38/17; A61N 1/30; A61K 9/14; A61K 31/7072; A61K 31/18

(52) U.S. Cl. 514/12; 604/20; 424/486; 514/49; 514/602

(57) ABSTRACT

Provided is a new therapeutic method to treat cancer that combines radiofrequency (RF) ablation and local controlled drug delivery. A preferred method utilizes localized drug delivery of 5-fluorouricil (5-FU) from polyanhydride implants.

### DOMESTIC PRIORITY CLAIM

This application claims the benefit of U.S. Provisional Application No. 60/374,036 filed Apr. 19, 2002.

### TECHNICAL FIELD

The present invention relates to a combination of ablation and controlled drug delivery for the treatment of cancer.

### BACKGROUND OF THE INVENTION

Cancer is currently the second leading cause of death and it is expected that in the near future it may exceed cardiovascular disease. Treatment of the disease depends upon the primary tumor cell type and its stage at the time of presentation. Cures are possible, however, the major focus of current cancer therapy is to improve the mortality and morbidity using palliative methods such as surgery, radiation therapy, and chemotherapy.

New innovative treatments have been attempted that, while offering great promise for improvement, nevertheless continue to have drawbacks. Local treatment for metastatic disease has become simplified and expanded to include local ablation using freezing or radiofreqency heating. A further benefit of the new methods of freezing and heat-coagulation is that these can be employed not only in the surgical arena, but also for percutaneous treatments. The benefits of the percutaneous image-guided methods include lower morbidity, reduced cost, and shortened hospital stay. The shortcoming of all methods is the incidence of tumor recurrence at the treated sites.

Multiple authors have evaluated the local injection of chemical agents (such as alcohol and acetic acid) for the destruction of hepatic tumors, as well as standard chemotherapeutic agents into focal tumors. Localized chemical treatment offers the advantage of very high local concentration of the agent with a low systemic or regional concentration, minimizing the risk of associated side effects. If sustained high local concentrations can be maintained then the advantages of time dependent sensitivities can be capitalized upon. The difficulties with liquid agents are the inconsistent spread when they are directly injected as fluids or gels. Furthermore, complications have occurred because of the diversion of the injected agents into the local and regional blood supply so that occlusion of vessels and massive necrosis of the liver have occurred. Several limited clinical models studies have shown some benefit to locally injected gel materials, but also confirmed the difficulty with variable distribution.

Recent studies report the use of local treatment with radiofrequency heat ablation. The reported advantages include shorter treatment times, smaller recurrence rate, lower morbidity, and the possibility to perform percutaneous treatments. Radiologic pathologic studies show that while the radiofrequency is quite effective centrally, residual tumor resides within the periphery of such lesions. Indeed the clinical experience has borne out the significance of this finding in that there is a well-defined recurrence rate with these methods and the site of recurrence is typically the periphery of the lesion, at the interface with the normal liver.

Thus, there is a continuing need for innovative approaches to cancer therapy. We provide here a new approach for tumor ablation that overcomes the disadvantages of the approaches applied previously. With this new method we combine ablation treatment of local disease with local controlled drug delivery. This combination offers great promise because it is believed that ablation kills a very high percentage of tumor cells and the local sustained release of drug completely eradicates residual tumor cells. A second important benefit is that the method of the present invention may reduce the incidence of needle tract tumor recurrences that have been reported with radiofrequency ablation, cryofreezing, and laparoscopy.

### BRIEF DESCRIPTION OF THE DRAWINGS

FIG. 1 shows the dissection scheme of rat liver samples.

FIG. 2 shows the release of 5-fluoruracil (5-FU) from poly[1,3-bis(carboxyphenoxy)propane-co-sebacic acid] (poly(CPP:SA)) implant in fetal bovine serum (FBS) and phospate buffered saline (PBS) at 37° C.

FIG. 3 shows the spatial distribution of 5-FU in radiofrequency (RF) ablated liver at 24 h and 48 h after the ablation and implantation.

FIG. 4 shows the spatial distribution of 5-floxuridine (5-FUR) in RF ablated liver at 24 h and 48 h after the ablation and implantation.

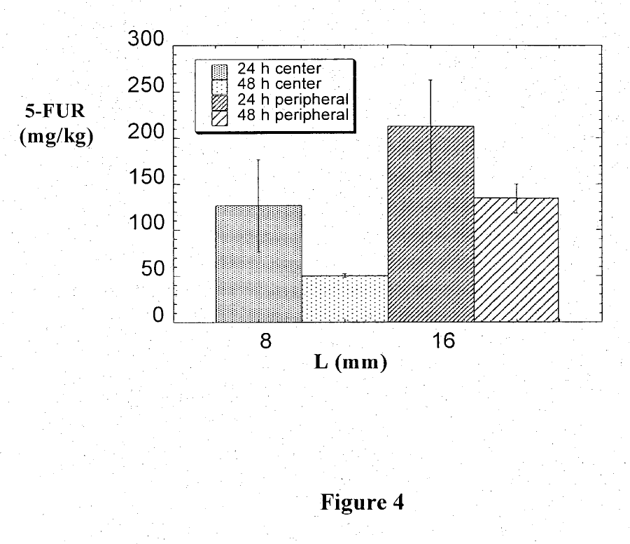

### SUMMARY OF THE INVENTION

In accordance with the present invention, there is provided a method for the treatment of cancer comprising ablation in combination with local controlled drug delivery of an active agent.

Any means for providing ablation is suitable for use in the method of the invention. Such techniques are known in the art and include radiofrequency ablation, microwave ablation and freezing. A preferred method utilizes radiofrequency ablation.

The local controlled drug delivery utilizes at least one biodegradable polymer implant containing at least one active agent. The biodegradable polymer can be selected from any of the well-known biodegradable polymers, e.g., polyanhydride, polyorthoester, or polylactide/glycolide, or combinations thereof. A preferred biodegradable polymer of use in the invention is a polyanhydride. Most preferred is a polyanhydride that is poly[1,3-bis(carboxyphenoxy)propane-co-sebacic acid].

Any active agent that can provide the intended therapeutic effect is suitable for use in the method of the invention. Preferred active agents are those that act as chemotherapeutic (chemotoxic or chemostatic) agents. More preferred chemotherapeutic agents are those of the antiproliferative, antiangiogenic, or vasoconstrictive classes. Most preferred is the chemotherapeutic agent 5-fluorouracil (5-FU).

The placement of the implant containing the active agent can occur prior to, simultaneously with, or following ablation. Preferred methods of the invention provide the implant after ablation, more preferably within one day following ablation.

The present invention also provides a method for the treatment of cancer comprising ablation in combination with local controlled drug delivery of at least one active agent wherein the local controlled drug delivery comprises the placement of at least one biodegradable polymer implant positioned around the site of the tumor lesion. A preferred feature of this aspect of the invention is the placement of the implant radially around the site of the tumor.

The present invention also provides a method for increasing the concentration of active metabolite of a locally administered drug in a desired tissue, comprising the thermal modification of the tissue. In this aspect of the invention, the area of the tissue modified by ablation treatment acts as a reservoir for the active agent. As the active agent diffuses from the reservoir, the agent is metabolized into its active form by the tissue unaffected by ablation, thereby increasing the concentration of active metabolite at the margins of the ablated tissue.

The present invention also provides a local controlled drug delivery device comprising an antiproliferative or antiangiogenic agent combined with a vasoconstrictive agent.

The present invention also provides a method for the treatment of cancer comprising ablation in combination with local controlled drug delivery of an active agent wherein the local controlled drug delivery comprises an antiproliferative, antiangiogenic or vasoconstrictive agent alone or in combination with one or more chemotherapeutic agents. Representative agents of this class include suramin or its analogs and endothelin, respectively. Agents such as these can suppress the growth of vasculature that may be naturally stimulated during regrowth of an ablated tumor. Tumors normally obtain a hypervascular blood supply on their outer regions and this vasculature may still be present in the peripheral regions following ablation. This enhanced zone of tumor blood flow can act as a sink to carry away any chemotherapeutic agent that may diffuse to these blood vessels. It is thus desirable to provide locally an agent that will constrict the blood vessels and slow down the wash-out of the chemotherapeutic agent. Suitable agents for this feature of the invention include epinephrine in combination with 5-fluorouricil and epinephrine in combination with cisplatin. Suitable vasoconstrictive agents include endothelin. Endothelin-1 is a very potent vasoconstrictor produced locally by the vascular endothelium that functions, together with nitric oxide, to modulate vascular tone. In studies done in livers, the sinusoidal circulation undergoes intense vasoconstriction with endothelin. Adding a slow release endothelin along with the slow release anti-tumor agent would constrict blood vessels in the immediate area and therefore facilitate the uptake of the anti-cancer agent and/or its active metabolite at the desired site.

### DETAILED DESCRIPTION OF THE INVENTION

As used herein, the singular forms “a”, “an”, and “the” include plural reference unless the context clearly dictates otherwise.

As used in the present specification, the following terms have the meaning indicated:

The term “ablation” refers to a tissue destructive technique whereby heating or cooling is applied to a local region by means of an instrument at such a level that cell viability is extinguished for the majority of the cells subjected to the process. Such instruments include a radiofrequency or microwave generator tuned to the vibration of water in the tissue and providing local heating. Instruments that deliver local freezing to tissue and result in local cell death also cause ablation.

The term “cancer” includes both precancerous and cancerous lesions.

The term “controlled release polymer implant” refers to a solid that can be placed in a tissue, the solid of which contains an active agent for delivery to that target region in a time-release manner. The implant can be made of but not limited to a biodegradable polymer. Suitable polymers include biocompatible polycaprolactones, polyhydroxybutyrates, polylactides and polylactaide-co-glycolides, polycarbonates, polyanhydrides, polyurethanes, polyacrylates and poly(ortho esters).

The term “active agent” refers to a drug that is delivered to a targeted site by means of a controlled release polymer implant. The drug provides a therapeutic benefit to a patient with a cancer. Such benefits include, but are not limited to the modification of cell proliferation, cell migration, vessel constriction, and inflammation.

Briefly, the combination of ablation and local controlled drug delivery has been demonstrated in both a rat model and a rabbit tumor model. In the rat model, radiofrequency ablation and the kinetics of localized 5-FU release kinetics has been demonstrated with bioresorbable polyanhydride implants. In the rabbit model, a similar methodology is employed in a hepatic VX2 tumor model.

In the preferred method the subject, having one or more focal lesions on an affected organ, is prepared for surgery and administered local or systemic anesthesia. Under computed tomography (CT) guidance, an appropriately sized needle is inserted through the skin and into the cancerous lesion within the diseased organ. The needle is removed and followed by the insertion of a RF probe. The probe administers RF radiation to heat the tissue of the lesion raising the temperature of the surrounding tissue to 90° C. or more. The position of the RF probe can be readjusted so as to ablate as much of the lesion as possible. Then the probe is removed and a trocar that contains at least one controlled release implant is inserted into the needle tract. The controlled release implant containing the active agent is placed in the center of the ablated lesion. After removal of the trocar, the pathway is sealed with a sterile medical grade gelfoam.

Variations of the preferred method can include ablation followed by a waiting period of at least one day before the implant is inserted. A second variant of the method could involve insertion of the implant followed by ablation. This may cause melting of the implant, a burst release of a portion of the drug and a resulting increase in its surface area of the implant upon cooling. For polyanhydrides, which are known to degrade by surface erosion, an increased drug release rate would be expected with increased surface area.

An important advantage of the present invention is that the release of active agent can be controlled for days or even weeks, the rapid washout of agent is eliminated and a sustained local effective dose is maintained. Controlled release implants can be made following techniques well known in the polymer drug delivery arts, e.g., by injection molding, and both the polymer content and drug loading can be adjusted to achieve the desired release profile. Another important feature of the present invention is that the implants can be designed to allow easy delivery into the ablation site through the probe insertion hole using a trocar.

Poly[1,3-bis(carboxyphenoxy)propane-co-sebacic acid] (Poly CPP:SA, 1:4 w/w) has been used, and is preferred, as a biodegradable carrier for 5-FU in the present invention. The United States Food and Drug Administration have approved polyanhydride polymers of similar composition for use as a biodegradable controlled release brain wafers to treat glioma using the alkylating agent, carmustine (BCNU).

A variety of chemotherapeutic agents can be formulated in a biodegradable matrix. For tumors of the liver and pancreas, 5-FU and gemcitabine are two of many possible choices. 5-FU is well characterized and has also shown beneficial effects in carcinomas of the breast, gastrointestinal tract, bladder, prostate, pancreas and oropharyngeal areas.

A preferred composition for the local controlled delivery of an active agent comprises a therapeutically effective amount of the active agent, delivered at the site of treatment in a sustained fashion. This can be determined based on the known release kinetics of the controlled release vehicle and the transport rate for active agent. For those vehicles for which a release profile is not known, one can determine the suitability of the vehicle following the teachings of the present invention. The drug transport rate of the active agent can be used to determine the transport away from the desired site(s) of treatment. For 5-FU it has been reported that a level of 0.05 μg/g in neoplastic tissue is sufficient for a clinical effect.

Our results show that the radiofrequency ablation combined with local polymer-based drug treatment resolves many of the shortcomings of methods described in the art. First, ablation apparently destroys the small vasculature within the ablation area so that the retention of the 5-FU is improved. The distribution of the primary agent is homogeneous within the site and around the site. Furthermore, because the normal liver around the site more rapidly processes the parent drug, the concentrations of the active metabolite are actually higher at the margins than in the compromised tumor. The margins are the most common site shown by pathologic and clinical studies to be the offending area where residual tumor cells are harbored and also recurrences result.

Another multimodality approach to retard or prevent the regrowth of a tumor after ablation is to use anti-angiogenic drugs such as suramin or its analogs. Drugs such as this can suppress the growth of a vasculature that may be naturally stimulated during regrowth of an ablated tumor. Tumors normally obtain a hypervascular blood supply on their outer regions. After ablation, this vasculature may still be present in the peripheral regions. This enhanced zone of tumor blood flow can act as a sink to carry away any anti-tumor agent that diffuse to these blood vessels. It is desirable to provide locally an agent that will constrict the blood vessels and slow down the wash-out of the anti-tumor agent. Such approaches have been reported with epinephrine in combination with 5-fluorouricil and epinephrine with cisplatin for local treatment of tumors using gels. Another agent that could be used as a combination drug with an antiproliferative is endothelin. Endothelin- I is the most potent vasoconstrictor in the body. It is produced locally by the vascular endothelium and modulates vascular tone along with nitric oxide. In studies done in livers, the sinusoidal circulation undergoes intense vasoconstriction with endothelin. Adding a slow release endothelin along with the slow release anti-tumor agent would constrict blood vessels in the immediate area and therefore facilitate the uptake of the anti-cancer agent and/or its active metabolite at the desired site.

The following examples will serve to further illustrate the method of the invention.

### EXAMPLE 1

### Preparation of Biodegradable Controlled Release Implants

The biodegradable polymer used for formulation and melt-extrusion of 5-FU-loaded implants was a 1:5 equivalent ratio copolymer of 1,3 bis(p-carboxyphenoxy propane) with sebacic acid. It had an apparent weight average and number average molecular weight of 108,000 and 24,000, respectively, as determined by gel permeation chromatography (GPC) against polystyrene standards. This polymer was prepared by the Specialty Products Division of Abbott Laboratories and was stored at −80° C.

All drug formulation and extrusion work was performed in an isolator chamber designed to provide minimal human exposure to oncolytic drugs. Drug blending into the polymer melt and extrusion of the formulated controlled release material was done using a bench scale (5 g mixing chamber) twin-screw compounder manufactured by DACA Instruments, Goleta, Calif. Before blending, the 5-FU was ground to a fine particle size using a mortar and pestle. The polymer was added first to the DACA compounder, which was set to a temperature 80° C. and a mixing speed of 140 rpm, followed by the 5-FU, added to a level of 15-20% by weight. After all of the drug had been added, the compounder was run for 2 minutes to ensure complete mixing. Then it was extruded onto a moving conveyer belt that could be set to produce strands which could range in diameter between 0.042″ and 0.025″ depending on belt speed. After solidification, the strands were cut into reasonable lengths and stored at low temperature. The cylindrical geometry of the implants was such that the desired dosage of drug could be obtained by cutting the implant to a predetermined length and could be administered percutaneously by use of a trocar. Placebo implants were made from the same polymer in the same way, but the drug was omitted during processing.

### EXAMPLE 2

### Analysis of Drug Content in Implant Material and In-Vitro Release

The drug 5-fluorouracil was analyzed by HPLC using a Regis C-18 reverse phase column. The mobile phase was deionized water at a flow rate of 0.6 ml/min and the injection volume was10 μl. The detector was set to monitor 266 nm. Calibration standards for the method were prepared as follows: A mass of 13 mg of 5-FU was dissolved in deionized water in a 50 ml volumetric flask to make a 2.00 mM stock solution. Sequential dilutions of the stock solution give standard solutions of 1.00, 0.200, 0.0500, 0.0100 and 0.00100 mM, respectively. The standards for the metabolite 5-FUR were similarly prepared at the concentrations of 2.00, 1.00, 0.200, 0.0500, 0.0100 and 0.00100 mM, respectively. The HPLC peak area of 5-FU and 5-FUR varied linearly with concentrations in the standard test range of 0.001-2 mM.

To measure in-vitro drug release of 5-F U from the implants, solid samples were pre-weighed and placed in a sterile 50-ml conical tubes filled with 20 ml of either phosphate buffered saline (PBS) or fetal bovine serum (FBS). The tubes were placed on the Gyromax 929 shaker adjusted to 37° C. and 120 rpm. Periodically, 0.1 ml of the solutions was transferred into a vial and diluted 20 times with deionized water. Aliquots of 0.1 ml of fresh PBS or FBS was added to the conical tubes each time to maintain a constant volume. The 20× diluted PBS samples were filtered through a 0.2 μm syringe filter, and used for HPLC analysis.

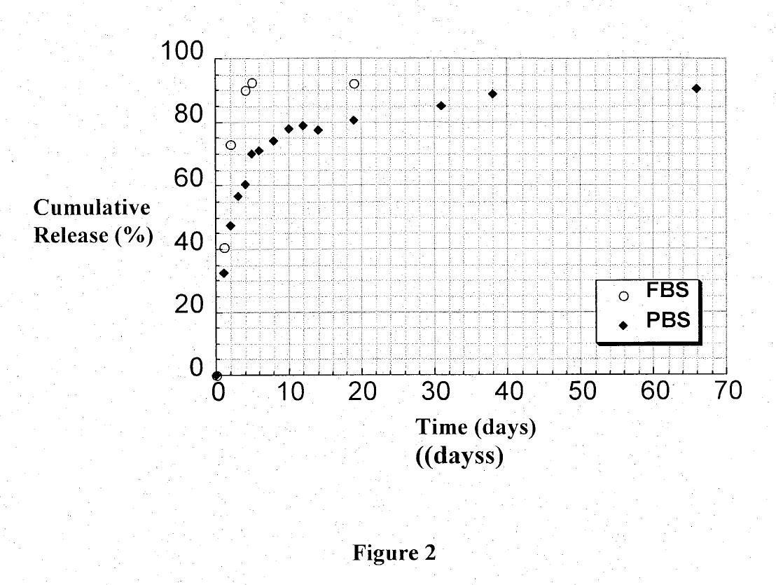

The drug concentration of the implants was determined to be 17%. This gives a total drug dosage of approximately 0.25 mg. In PBS, 75% of the 5-FU was released in 9 days, while it only took 2 days to reach that point in FBS (FIG. 2).

### EXAMPLE 3

### Implantation of 5-Fluorouracil Rods in the Livers of Healthy Rats

Two male Sprague Dawley rats, 350-450 g, were anesthetized with an intraperitoneal injection of sodium pentobarbital (50 mg/kg, Abbott, Chicago, Ill.). The abdomens were prepared and Marcaine (Abbott, Chicago, Ill.) was injected subcutaneously just prior to the skin incision. The medial lobe of the livers were exposed through a small midline incision and exteriorized for placement of the rods. The capsule and parenchyma of the livers were punctured with a 23-gauge needle and tracts made.

The rods for implantation measured approximately 1.5 mm in diameter by 5 mm long. They were inserted into the tracts made by the needles. A small piece of fat was placed over the insertion sites for the rods and sewn to the capsule of the liver. The abdomens were sutured closed and the animals were allowed to recover. One animal was sacrificed after 24 hours and the second after 48 hrs. The livers were removed and immediately frozen for the analysis of 5 FU and its metabolites as described.

### EXAMPLE 4

### Ablation and Implantation of 5-Fluorouracil (5-FU) Rods in the Livers of Healthy Rats

Two male Sprague Dawley rats, 350-450 g, were anesthetized with an intraperitoneal injection of sodium pentobarbital (50 mg/kg, Abbott, Chicago, Ill.). The abdomens were prepared and Marcaine (Abbott, Chicago, Ill.) was injected subcutaneously just prior to the skin incision. The medial lobe of each liver was exposed through a small midline incision.

For the ablation procedure, the livers were placed on a wire mesh grounding pad and a saline soaked sponge. Ablation was accomplished using a 200-W radiofrequency generator (model CTRF-A, Radionics, Burlington, Mass.) using a 19-gauge needle electrode (model PE-D(10)(2) Radionics , Burlington, Mass.). Ablation was initiated by inserting the needle electrode into a needle tract made with a 23-gauge needle in the center of the lobe. The electrode was inserted to a depth of approximately 8-mm. The hepatic tissue exposed to the radiofrequency current was raised to a temperature of 90° C. for 3 min. This approach resulted in an ablated area of approximately 1-cm. After the electrode was removed, a 5-FU rod was placed in the probe tract and a fat pad was sewn over the top. The abdomens were sutured closed and the animals were allowed to recover. One animal was sacrificed after 24 hours and the second after 48 hrs. The livers were removed and immediately frozen for the analysis of 5-FU and its metabolite 5-FUR as described. It was noticed that as a result of RF ablation a clear circular boundary exists between dead and living tissue.

### EXAMPLE 5

### Measurement of the Local drug Concentration distribution in Rat Livers

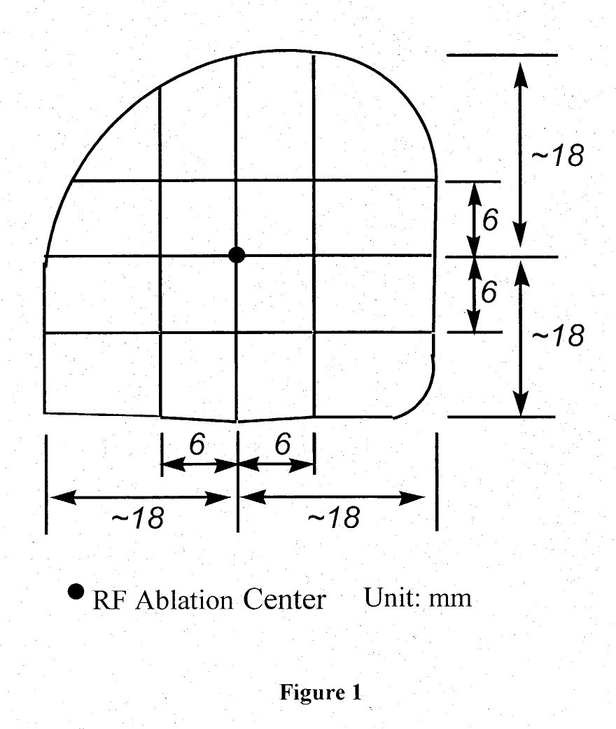

The livers were dissected into analysis samples according to FIG. 1. The individual liver samples were transferred into a 1.5-ml microtubes and homogenized with a Kontes pellet tissue grinder for 3 min. Then 1.0 ml of deionized water was added to each homogenized sample, and the tubes were shaken at 240 rpm for 12 h. Three drops of 10% HClO₄ was added to each tube, and the tubes were shaken for an additional 1 h. The microtubes were then centrifuged and the supernatant was filtered through a 0.2 μm syringe filter. The filtered solutions were used for HPLC analysis of the drug concentration and the tissue concentrations of 5-FU and 5-FUR were calculated.

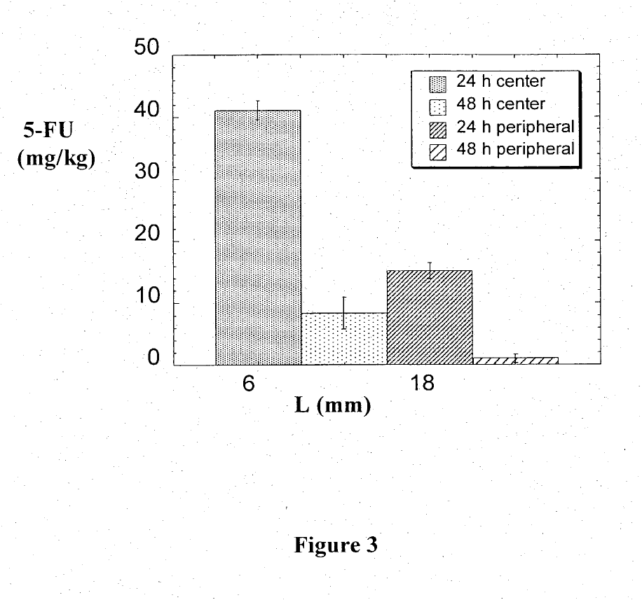

The average diameter of the ablated area was ˜1 cm; however, 5-FU is distributed throughout the whole liver (and possibly beyond). Within 24 h, the 5-FU concentration in the immediate vicinity of the ablation site (˜6 mm radius) reaches as high as 41 mg/kg of liver tissue. Between 6 and 18 mm away from the ablation center the concentration approaches that of typical systemic dose level of 12 mg/kg. Such a local effective distribution should maximize the effect on cancer cells, while at the same time minimize the damage to normal tissue. The distribution of 5-FU metabolite, 5-FUR, is different from that of 5-FU in ablated livers. The immediate vicinity of the ablation center actually contains less 5-FUR than that further away from the ablation site. This is likely caused by a lack of enzymatic activity in the ablation site. These results are shown in graphical form in FIGS. 3 & 4 for 5-FU and 5-FUR, respectively.

In sharp contrast to this is the distribution of 5-FU and 5-FUR in normal (unablated) liver (see example 3). In 24 h, the concentration of 5-FU and 5-FUR is essentially the same throughout the whole liver. No significant difference is observed between the insertion point and the periphery of the liver. This presumably is due to the relatively intact circulatory system of the liver.

### EXAMPLE 6

### Implantation of Fresh VX-2 Tumor in the Liver of a Healthy Rabbit

Male New Zealand white rabbits (−3 kg) were used and were cared for in accordance with the Case Western Reserve University animal research center regulations. The rabbits were anesthetized using a combination of xylazine (5 mg/kg body weight BW) and ketamine (50 mg/kg BW) i.m. injection. The abdomen was shaved and cleaned with povidone-iodine solution (Betadine), and 0.5 ml of Marcaine was administered into the skin at the incision site. A small midline incision was made and the medial lobe of the liver was exposed and exteriorized. The capsule of the medial lobe was punctured with a scalpel blade and a small piece of VX-2 tumor (1 mm³) recovered 30 min prior to the implantation procedure was inserted into the subcapsular space. A small piece of fat was sutured over the tumor implant. The abdominal muscles were closed with suture and the skin was closed with subcutaneous hidden sutures. During the recovery period, all animals received the pain relieving agent buprenophrine (Buprenex, 0.5 mg/kg, i.m.) and once again 24 hrs later. If the animals were perceived to be in pain, additional buprenophrine was available if needed. The rabbits also received a subcutaneous injection of 5% dextrose in saline during recovery and 24 hrs later. Tumor growth was monitored by CT every 3 to 5 days. After 35 days, the size of the tumor was approximately 4-6 cm in size and had infiltrated the whole medial lobe with a lot of necrosis observed in the center. Metastases to the lung were also noted when the animal was euthanized.

### EXAMPLE 7

### Implantation of Frozen VX-2 Tumor in the Liver of Healthy Rabbits

The procedure of Example 6 was repeated in an additional rabbit but instead of implanting fresh VX-2 tumor, VX-2 tumor which had been harvested earlier from a rabbit in which the tumor was implanted in the thigh for propagation and frozen for storage was used. One rabbit was sacrificed at 13 days. The tumor size as measured by CAT scan was 18 mm×16 mm×22 mm.

### EXAMPLE 8

### Implantation of Frozen VX-2 Tumor Plus One Controlled Release Rods Containing 5-FU in the Liver of Healthy Rabbits

The procedure of Example 7 was repeated in an another rabbit but in addition to placement of VX-2 tumor that has been frozen previously, a 5-FU-contolled release rods of polyanhydride, 7 mm long, was placed under the tumor implantation site using a similar approach as described for the rat in Example 3. At 15 days the tumor grew to 13×11×13 mm, a size smaller than observed in Example 7, which had no implanted 5-FU rod. At autopsy, the tumor also appeared smaller, more discrete in size and surrounded by a well defined capsule. This appearance was less diffuse than the tumor observed in the untreated rabbit in Example 6.

### EXAMPLE 9

### Implantation of Frozen VX-2 Tumor plus Three Controlled Release Rods Containing 5-FU in the Liver of Healthy Rabbits. Tumor Measurement After 39 Days.

The procedure of Example 7 was repeated on an additional rabbit but in addition to placement of the VX-2 tumor that which had been frozen previously, three 5-FU-contolled release rods of polyanhydride measuring approximately 7 mm in length were placed circumferentially around the tumor implantation site at the time of tumor implantation as described in Example 8. At 39 days, a tumor was not readily discernable by CAT scan.

### EXAMPLE 10

### Implantation of Frozen VX-2 Tumor plus Five Controlled Release Rods Containing 5-FU in the Liver of Healthy Rabbits. Tumor Measurement After 41 Days.

The procedure of Example 8 was repeated on an additional rabbit. In this protocol, five 5-FU polyanhydride rods were placed around the VX-2 tumor implantation site. At 41 days, a measurable increase in the size of the tumor since implantation was not measurable.

### EXAMPLE 11

### Percutaneous Implantation of Controlled Release Rods Containing 5-FU into the VX-2 Tumors in Rabbits

The optimal time for imaging, RF ablation and implantation of polymer millirods is 10-15 days; thereafter the tumors tend to become large and centrally necrotic. The controlled release rod implantation procedure is as follows. A 14 Gauge biopsy needle is placed percutaneously into the VX-2 liver tumors under CT guidance. Prior to inserting this needle, the abdominal site is infiltrated with Mercaine (0.5 cc) to prevent any distress. The cutting blade is positioned across the tumor tissue and defines the implantation site of the polymer millirod. The stylette is removed, and a polymer millirod containing 5-fluorouracil is inserted and placed inside the tumor tissue.

### EXAMPLE 12

### Percutaneous Ablation and Implantation of Controlled Release Rods Containing 5-FU into RF Ablated VX-2 Tumors in Rabbits

The animal is prepared and anesthetized as described in Example 5 in the tumor implantation step. Ablation was accomplished using a 200-W radiofrequency generator (model CTRF-A, Radionics, Burlington, Mass.) as follows: The liver capsule of the medial lobe is pierced with an 18-gauge hypodermic needle, and the liver tissue is ablated with a 19-gauge needle electrode (model PE-D(10(2)-K, Radionics®, Burlington, Mass.), at 90±3° C. for 3 minutes. Small segments (0.7-0.8 cm in length) of the 5-fluorouracil implants are then implanted using a biopsy needle into the ablated tumor. Following insertion of the rod, a piece of gelfoam is pushed into the pathway to seal the pathway site.

### EXAMPLE 13

### Intraoperative Ablation and Implantation of Controlled Release Rods Containing 5-FU into RF Ablated VX-2 Tumors in Rabbits

The animals are prepared and anesthetized as described in Example 6 in the tumor implantation step. The liver is exposed through an incision in the abdomen and placed directly on the RF ablation grounding pad. Ablation is accomplished using a 200-W radiofrequency generator (model CTRF-A, Radionics, Burlington, Mass.) as follows: The liver capsule of the medial lobe is pierced with an 1 8-gauge hypodermic needle, and the liver tissue is ablated with a 19-gauge needle electrode (model PE-D(10)(2)-K, Radionics®, Burlington, Mass.), at 90±3° C. for 3 minutes. Small segments (0.7-0.8 cm in length) of the 5-fluorouracil implants are then implanted into the ablated tumor of the “test set” of rabbits. Small segments (0.7-0.8 cm in length) of the placebo polymer implants containing no drug are then implanted into the ablated tumor of the “control set” of rabbits. A small piece of fat is sutured on top of the implantation sites to seal the sites. The abdomens are closed with sub-cutaneous vicryl sutures to prevent irritation to the animal. After 15 days or more one sees that the tumor recurrence rate and average size of new tumors are smaller than that of the control group.

### EXAMPLE 14

### Implantation of 5-FU Rods Plus Radio-Frequency Ablation in VX-2 Tumors in the Liver of Rabbits

Male New Zealand white rabbits (−3 kg) were used and were cared for in accordance with the Case Western Reserve University animal research center regulations. The rabbits were anesthetized using a combination of xylazine (5 mg/kg body weight BW) and ketamine (50 mg/kg BW), acepromazine (2 mg/kg), and atropine (0.2 mg/kg) given by i.m. injection. The abdomen was shaved and cleaned with povidone-iodine solution (Betadine) and 0.5 ml of Marcaine was administered into the skin at the incision site. A small midline incision was made and the medial lobe of the liver was exposed and exteriorized. The capsule of the medial lobe was punctured with a scalpel blade and a small piece of VX-2 tumor (1 mm³), which was propagated in another rabbit and stored in liquid nitrogen for later use, was inserted into the liver parenchyma. A small piece of fat was sutured over the implant site. The abdominal muscles were closed with suture and the skin was closed with subcutaneous hidden sutures. During the recovery period, all animals received buprenophrine (Buprenex, 0.5 mg/kg, i.m.), soon after surgery and once again 24 hrs later for pain. If the animals were later perceived to be in pain, additional buprenophrine was given as needed. The rabbits also received a subcutaneous injection of 25 ml of 0.9% saline during recovery and 24 hrs later.

Fourteen days after tumor implantation, the rabbits were anesthetized again using the protocol given above. The medial lobe of the liver was exteriorized and examined for tumor growth. The size of the tumor was approximately 0.5-1.0 cm in diameter and extended into the liver parenchyma. Before radio frequency ablation, a grounding pad was placed under the exteriorized medial lobe of the liver. Ablation was accomplished using a model CTRF, 200 watt radiofrequency generator equipped with a 19-gauge needle electrode, model PED(10)(2)K, both manufactured by Radionics, Burlington, Mass. The tumor was pierced with the electrode and the tissue was ablated at 90±3° C. for 5 minutes. In one control animal, the tumor was not ablated at 14 days and served as a non-ablated, non-5-FU rod treated control.

Following ablation, a subgroup of these animals received implantation of 5-FU rods. For the ablation-only treated subgroup, a fat pad was placed over the ablation sites and the animals were allowed to recover as described. For the ablation plus 5-FU rod implantation subgroup, three rods, each measuring approximately 7 mm long by 0.5 mm diameter were placed together into the hole created by the needle electrode. A fat pad was then sutured over this area and the animals were allowed to recover as described. At 14 days CT scans of the livers were taken and the animals were then terminated. The livers with tumors were removed and photographed with a digital camera before placing them in formaldehyde for fixing. The abdomen and lungs were also photographed and lung samples taken for histological analysis.

Tumor volumes after 28 days of growth, calculated from the digital photographs as well as the integration of the CT slices though the tumors of each rabbit, are given in the table below:

Tumor Volumes (cm³), mean, (1 SD)

| Treatment | Digital Photography | CT Scan | p value |
|---|---|---|---|
| No Treatment (N = 1) | 8.1 | not done | |
| Ablation-only (N = 5) | 2.41 (0.55) | 3.92 (0.94) | <0.01 |
| Ablation + 5-FU (N = 4) | 1.24 (0.55) | 1.71 (0.56) | <0.01 |

In addition to the approximate 50% reduction in tumor size in the livers that received local sustained release 5-FU, those animals also appeared to fare better than the ablation only subgroup. The ablation only subgroup exhibited a 4.5% weight loss over the last 14 days following the RFA intervention versus a 2% increase in body weight in the RFA plus 5-FU subgroup.

The foregoing is only illustrative of the invention and is not intended to limit it the invention to the disclosed embodiments. Variations and changes that are obvious to one skilled in the art are intended to be within the scope and nature of the invention that are defined in the appended claims.

We claim:

1. A method for the treatment of cancer comprises ablation in combination with local controlled drug delivery.

2. The method of claim 1 wherein the ablation is radiofrequency ablation.

3. The method of claim 1 wherein the local controlled drug delivery comprises at least one biodegradable polymer implant containing at least one chemotherapeutic agent.

4. The method of claim 3 wherein the biodegradable polymer is selected from polyanhydride, polyorthoester, or polylactide/glycolide.

5. The method of claim 3 wherein the chemotherapeutic agent is selected from the groups comprising antiproliferative, anti-angiogenesis, or vasoconstrictive agents.

6. The method of claim 5 wherein the chemotherapeutic agent is 5-FU.

7. The method of claim 5 wherein the chemotherapeutic agent is sumarin or one of its analogs.

8. The method of claim 5 wherein the chemotherapeutic agent is endothelin-1.

9. The method of claim 4 wherein the biodegradable polymer is polyanhydride.

10. The method of claim 9 wherein the polyanhydride is poly[1,3-bis(carboxyphenoxy)propane-co-sebacic acid].

11. The method of claim 3 wherein the local controlled drug delivery comprises more that one biodegradable polymer implant radially positioned around the site of the tumor lesion.

12. A method for increasing the concentration of active metabolite of a locally administered drug in a desired tissue, comprising the thermal modification of adjacent tissue providing a benign reservoir for such drug.

13. An implant containing an antiproliferative combined with a vasoconstrictive agent.

14. The method of claim 1 where the implant is added to the ablation site as least 1 day after the ablation procedure.

15. The method of claim 1 where the implant is inserted into the tumor prior to the ablation process.
